SEQUENCE LISTING (5 sequences)

SEQ ID NO: 1 - DNA, 39 bp, synthetic construct
  source 1..39: mol_type = other DNA; note = primer A
  modified_base 1: mod_base = OTHER; note = acrylamide phosphoramidite modification at 5' end
  modified_base 3: mod_base = OTHER; note = Uracil
  misc_difference 19..24: note = n=a, c, g, or t/u
cctcctactctgacgtcgnnnnnnggtaccttgtcccca

SEQ ID NO: 2 - DNA, 46 bp, synthetic construct
  source 1..46: mol_type = other DNA; note = primer B
  modified_base 1: mod_base = OTHER; note = acrylamide phosphoramidite modification at 5' end
acaataagctctatccacgatatagttcctcctttcagcaaaaaac

SEQ ID NO: 3 - DNA, 17 bp, synthetic construct
  source 1..17: mol_type = other DNA; note = primer C
  modified_base 1: mod_base = OTHER; note = Azide moiety modification at 5' end
accgcggtctattactg

SEQ ID NO: 4 - DNA, 26 bp, synthetic construct
  source 1..26: mol_type = other DNA; note = primer D
gcgaaattaatacgactcactatagg

SEQ ID NO: 5 - protein, 17 aa, synthetic construct
  source 1..17: note = spytag peptide
  MOD_RES 1: note = Dibenzocyclooctyne (DBCO) conjugation
CRGVPHIVMVDAYKRYK

(19) United States
(12) Patent Application Publication
(10) Pub. No.: US 2025/0163406 A1
(43) Pub. Date: May 22, 2025

(54) METHODS FOR GENERATING NUCLEIC ACID ENCODED PROTEIN LIBRARIES AND USES THEREOF

(71) Applicants: CZ BIOHUB SF, LLC, San Francisco, CA (US); THE REGENTS OF THE UNIVERSITY OF CALIFORNIA, Oakland, CA (US)

(72) Inventors: Adam Abate, Oakland, CA (US); Cyrille L. Delley, Oakland, CA (US)

(21) Appl. No.: 18/834,772

(22) PCT Filed: Feb. 6, 2023
(86) PCT No.: PCT/US23/62029

§ 371 Date: Jul. 31, 2024

Related U.S. Application Data
(60) Provisional application No. 63/307,249, filed on Feb. 7, 2022.

(51) Int. Cl. C12N 15/10; C12Q 1/6806; C12Q 1/6874

(52) U.S. Cl. CPC C12N 15/1075; C12N 15/1093; C12Q 1/6806; C12Q 1/6874

(57) ABSTRACT

The present disclosure provides materials and methods for identifying biomolecules from single cells and binding partners of the biomolecules using barcoded substrates such as beads. Provided herein are methods that enable generation of hundreds to billions of different molecular binders and simultaneous tagging with a molecular beacons are provided. The methods provided herein allow quick assembly of molecular probe libraries which can be used, in various embodiments, for highly multiplexed identification of molecular signatures and corresponding binder pairs.

### CROSS-REFERENCE TO RELATED APPLICATIONS

The present application claims priority to U.S. Provisional Patent Application No. 63/307,249, filed on Feb. 7, 2022, the entirety of which is incorporated by reference herein.

### STATEMENT REGARDING FEDERALLY SPONSORED RESEARCH

This invention was made with government support under grants U01 AI129206, AR068129, and RO1 HG008978 awarded by The National Institutes of Health. The government has certain rights in the invention.

### REFERENCE TO THE SEQUENCE LISTING

This application includes a sequence listing submitted electronically, in a file entitled “56903_Seglisting.xml,” created on Feb. 1, 2023 and having a size of 9,147 bytes, which is incorporated by reference herein.

### FIELD

The present disclosure relates generally to methods for the construction of nucleic acid encoded molecular binders and their uses.

### BACKGROUND

Molecular binders such as antibodies and other protein scaffolds form highly specific stable interactions with target molecules and surfaces. This property is widely exploited in precision medicine, such as cancer immunotherapy, diagnostic methods and molecular detection applications. These applications generally require two steps: First, a particular molecular binder with specificity for a target of interest must be identified and characterized. Second, the identified binder is charged with a payload, such as a drug, or with molecular beacons that link the binder identity to a signal which can be detected unambiguously (e.g., using fluorophores for spectroscopic detection, or DNA tags for detection by sequencing). This general procedure has several drawbacks. First, considerable knowledge about the biological specimen must be available in order to identify suitable target molecules and their corresponding binders. For example, for a successful tumor immunotherapy, cancer cells need to express a known characteristic molecular signature which is absent from healthy tissue in order to prevent off-target toxicity. Molecular binders against this signature must then be identified and modified which is time consuming and costly.

### SUMMARY OF THE INVENTION

One embodiment of the present disclosure provides a method for the construction of nucleic acid encoded protein libraries. Other embodiments of the present disclosure provide methods to use such libraries for the discovery of biomarkers and specific molecular binders for these biomarkers.

In one embodiment, the present disclosure provides a method of identifying a biomolecule from a single cell and a binding partner of said biomolecule, said method comprising the steps of: (a) preparing an expression construct comprising (i) a nucleic acid sequence encoding a biomolecule-binding protein, and (ii) a nucleic acid sequence encoding a binding domain capable of binding to a binding partner; (b) attaching the expression construct of (a) to a solid substrate, thereby forming an expression construct-substrate complex, wherein said substrate comprises a nucleic acid barcode and the binding partner, thereby forming an expression construct-substrate complex; (c) isolating the expression construct-substrate complex of (b); (d) incubating the isolated expression construct-substrate complex under conditions that allow (i) transcription and translation of the biomolecule-binding protein and the binding domain, and (ii) binding of the binding domain to the binding partner on the substrate, thereby labelling the biomolecule-binding protein with the nucleic acid barcode; (e) releasing the labeled biomolecule-binding protein of (d) from the substrate and contacting the labeled biomolecule-binding protein with a sample comprising a plurality of cells under conditions that allow binding of the biomolecule-binding protein to a biomolecule on a single cell; (f) encapsulating a single cell bound by the labeled biomolecule-binding protein of (e) in a droplet; and (g) sequencing the nucleic acid barcode of the labeled biomolecule-binding protein; thereby identifying a biomolecule from a single cell and a binding partner of said biomolecule.

In another embodiment, the isolating in step (c) comprises encapsulating in a droplet. In still another embodiment, the (i) nucleic acid sequence encoding the biomolecule-binding protein and/or the (ii) nucleic acid encoding the binding domain is a DNA sequence, and wherein (i) and (ii) are operably linked to a promoter. In yet another embodiment, the nucleic acid sequence encoding the biomolecule-binding protein and/or the nucleic acid encoding the binding domain is an RNA sequence.

In other embodiments, an aforementioned method is provided wherein the biomolecule-binding protein is selected from the group consisting of an antibody, a nanobody, a T cell receptor, a B cell receptor, and an antibody mimetic. In still other embodiments, the binding domain is selected from the group consisting of a protein, a polypeptide, a peptide, a non-natural amino acid, an aptamer, a nucleic acid sequence, a nucleoside analog, or a functional fragment thereof. In another embodiment, the binding domain is a peptide. In one embodiment, the binding domain is a Spycatcher protein and the binding partner is a Spytag peptide. In yet another embodiment, the binding domain is streptavidin or neutravidin or fragment thereof and the binding partner is biotin, a biotin analog or peptide with affinity for streptavidin and neutravidin.

The present disclosure also provides, in some embodiments, an aforementioned method wherein the expression construct further comprises a unique DNA barcode. In still other embodiments, the expression construct further comprises a linker nucleic acid sequence between the nucleic acid sequence encoding the biomolecule-binding protein, and the nucleic acid sequence encoding a binding domain. In other embodiments, the expression construct is selected from the group consisting of a plasmid, a cDNA, a DNA fragment, a RNA and a mRNA, or functionally equivalent molecules comprised of nucleic acids and nucleic acid analogues. In yet other embodiments, the solid substrate is a bead, a hydrogel bead, a microarray, a cell, a fixed cell, a cell fragment, a virus, a protein complex, a ribosome, a microparticle, a nanoparticle, a micelle, a liposome, a droplet, and a polymer.

In still other embodiments, an aforementioned method is provided wherein the sample comprising the plurality of cells is selected from the group consisting of a tumor sample, a tissue sample, a blood sample, an environmental sample, a microbial sample, a bacterial sample, a viral sample, and mixtures thereof. In other embodiments, the biomolecule is an antigen, a tumor antigen, a protein, a cell surface protein, a receptor, a hapten, a post translational protein modification, a glycan, a peptide, a permeabilized cell, and a virus, and fragments of any of the above.

In another embodiment, the present disclosure provides a method of determining a phenotype of a single cell comprising the steps of: (a) preparing an expression construct comprising (i) a nucleic acid sequence encoding a biomolecule-binding protein, and (ii) a nucleic acid sequence encoding a binding domain capable of binding to a binding partner; (b) attaching the expression construct of (a) to a solid substrate, thereby forming an expression construct-substrate complex, wherein said substrate comprises a nucleic acid barcode and the binding partner, thereby forming an expression construct-substrate complex; (c) isolating the expression construct-substrate complex of (b); (d) incubating the isolated expression construct-substrate complex under conditions that allow (i) transcription and/or translation of the biomolecule-binding protein and the binding domain, and (ii) binding of the binding domain to the binding partner on the substrate, thereby labelling the biomolecule-binding protein with the nucleic acid barcode; (e) releasing the labeled biomolecule-binding protein of (d) from the substrate and contacting the labeled biomolecule-binding protein with a sample comprising a plurality of cells under conditions that allow binding of the biomolecule-binding protein to a biomolecule on a single cell; (f) encapsulating a single cell bound by the labeled biomolecule-binding protein of (e) in a droplet; and (g) sequencing the nucleic acid barcode of the labeled biomolecule-binding protein; thereby determining a phenotype of a single cell.

In still another embodiment, an expression construct-substrate complex is provided comprising: (a) at least one expression construct comprising (i) a nucleic acid sequence encoding a biomolecule-binding protein, and (ii) a nucleic acid sequence encoding a binding domain capable of binding to a binding partner; and (b) a solid substrate comprising a nucleic acid barcode and the binding partner.

### BRIEF DESCRIPTION OF THE DRAWINGS

FIGS. 1A-1E show an exemplary workflow for one method described herein. FIG. 1A) Expression construct, consisting of a unique DNA barcode (I), a promoter sequence (II), a molecular binder domain (e.g., an antibody fragment) (III), a linker domain (IV) and a binding domain for tagging (e.g., a SpyCatcher protein) (V). FIG. 1B) Encapsulating a solid substrate (VII) with a single member of an expression construct library in a droplet (VI) and performing a digital droplet PCR using primers immobilized on the solid substrate, allows to cover the solid substrate with isogenic copies of the barcode and the library member. FIG. 1C) These solid substrates are then collected and re-encapsulated with an in vitro transcription translation mix (IVTT) in a new droplet. FIG. 1D) The IVTT transcribes and translates the immobilized gene which and creates millions of copies of the same library member as proteins (FIG. 1E, VIII). The binding domain (V) engages and binds the barcode sequence provided by the solid substrate. These barcodes are then released from the solid substrate to release the proteins into the supernatant from where they can be harvested and used for instance to stain cells. This process allows to create millions of protein copies from millions of library members, all uniquely encoded with a protein specific DNA tag.

FIG. 2 shows the preparation of an expression construct and protein expression thereof. Left: A green fluorescent protein (GFP) encoding expression construct was immobilized on polyacrylamide hydrogel beads through digital droplet PCR. Successful immobilization is visualized through hybridization of Cy5 fluorophore labeled reverse primer. Right: Encapsulation of these beads together with IVTT and incubation for 4 h at 30° C. yields correctly folded GFP, which is evident from the fluorescence profile of the droplets.

FIGS. 3A-3B show self-labeling of molecular binders with DNA barcodes. FIG. 3A) Protein Bioanalyzer Gel of the IVTT expression of a nanobody-SpyCatcher fusion protein construct. Lane I shows the protein marker, lane II the IVTT protein expression after 3 hours of expression and after removal of the ribosomes; lane III shows the same expression mix after adding a SpyTag-DNAbarcode fusion construct and 15 minutes of incubation time. The nanobody-SpyCatcher protein construct (lower arrow) automatically forms a covalent bond with the SpyTag-DNAbarcode causing an up shift in mass (upper arrow). This demonstrates that these constructs can undergo self-labeling in droplets. FIG. 3B) Three different CD4 specific nanobodies have been fused to SpyCatcher and expressed with an IVTT mix. The constructs have been labeled with a Cy5 fluorophore coupled to the SpyTag peptide. The plot shows a FACS profile of Jurkat cells stained with the three nanobody constructs (V) and negative controls (IV). This demonstrates that these constructs can be used to label cells.

### DETAILED DESCRIPTION

The present disclosure addresses the aforementioned shortcomings by providing compositions and methods to identifying biomolecules from single cells and binding partners of the biomolecules using barcoded substrates such as beads. As described herein, methods that enable generation of hundreds to billions of different molecular binders and simultaneous tagging with a molecular beacons are provided. The methods provided herein allow quick assembly of molecular probe libraries which can be used, in various embodiments, for highly multiplexed identification of molecular signatures and corresponding binder pairs. The compositions, including constructs and complexes provided herein, have applications in, for example, immunotherapy and single-cell phenotypic profiling.

The methods described herein provide a streamlined method to generate barcoded molecular binders which results in a massive reduction in cost per probe and enables the assembly of universal probe panels (e.g., panels featuring a specific binder for all possible targets in any situation). These panels allow simultaneous identification of molecular signatures on singe cells, together with the identity of the corresponding molecular binder. These binders can then be employed in targeted therapies.

Numerous applications of the compositions, including the expression constructs and construct-substrate complexes, are contemplated herein. For example, in one embodiment, the barcoded probe library generated according to the methods described herein can be used to measure molecular spectra of single cells, such as protein expression and glycan structure. Similar to single-cell RNA sequencing, these measurements allow investigators to deconvolute phenotype diversity in biological samples. In contrast to mRNA measurements, the signatures identified herein are actionable targets for clinical intervention. Furthermore, the methods further enable contemporaneous identification of the corresponding probe that can be used for subsequent targeting of these cells. These probes can then be used, for example, for preparing a CAR-T receptor or antibody-based drug to target aberrant cells in diseases such as cancer, autoimmunity and bacterial infections. In still another embodiment, methods provided herein can be used to identify antibody-antigen pairs in a massively multiplexed way. This allows a rapid way to generate an antibody with desired binding properties.

There exist currently two general methods to link proteins to nucleic acid tags: 1) Chemical linkage, which is low throughput, costly and time consuming. But yields high yields of tagged product; and 2) Display techniques such as phage display, bacterial or yeast display, mRNA and ribosome display are high-throughput capable of generating protein libraries cheaply but they are limited in the achievable construct yields by the stability and linkage yield of the display molecule (mRNA, ribosome and DNA display) or their solubility limit (viral or cell display).

However, a high library diversity is key to provide a specific binder for most or all possible biological targets and a high molecular binder yield is required to shift the binding equilibria towards complex formation. None of the current technologies achieve both and hence do not enable above mentioned applications. The present compositions, including constructs and complexes, and methods described herein provide means to prepare a co-migrating particle.

As will be appreciated by those of skill in the art, following the library preparation as described herein, numerous downstream analytic techniques can be applied, including sequencing methods. Sequencing reactions and techniques are well known in the art and include, without limitation, scRNA-seq, scDNA-seq, Ab-seq, ATAC-seq, cut and run sequencing, and cut and tag sequencing.

As used herein, the term “sample” or “biological sample” or “tissue sample” encompasses a variety of sample types obtained from a variety of sources, which sample types contain biological material. For example, the term includes biological samples obtained from a mammalian subject, e.g., a human subject, and biological samples obtained from a food, water, or other environmental source, etc. The definition encompasses blood and other liquid samples of biological origin, as well as solid tissue samples such as a biopsy specimen or tissue cultures or cells derived therefrom and the progeny thereof. The definition also includes samples that have been manipulated in any way after their procurement, such as by treatment with reagents, solubilization, or enrichment for certain components, such as polynucleotides. The term “sample” or “biological sample” encompasses a clinical sample, and also includes cells in culture, cell supernatants, cell lysates, cells, serum, plasma, biological fluid, and tissue samples. “Sample” and “biological sample” includes cells, e.g., bacterial cells or eukaryotic cells; biological fluids such as blood, cerebrospinal fluid, semen, saliva, and the like; bile; bone marrow; skin (e.g., skin biopsy); and viruses or viral particles obtained from an individual. In one embodiment the tissue sample is a frozen tumor tissue sample. In another embodiment, the sample can be comprised of a cell line that is, for example, grown under tissue culture conditions. In still other embodiments, the sample comprises an environmental sample, a microbial sample, a bacterial sample, a viral sample, and mixtures thereof. Additionally, the sample can comprise fixed cells, permeabilized cells (e.g., associated with solid substrates).

In still another embodiment, rather than a single cell, the methods described herein can be used to identify a biomolecule from a particle (e.g., virus or library on library screens where an expression library targets the solid substrate-immobilized constructs of a second library) and a binding partner of said biomolecule.

As described more fully herein, in various aspects the subject methods may be used to detect a variety of components or “biomolecules” from such biological samples. Biomolecules of interest include, but are not necessarily limited to, polynucleotides (e.g., DNA and/or RNA), polypeptides (e.g., peptides and/or proteins), and many other components that may be present in the sample. In some embodiments, the biomolecule is an antigen, a tumor antigen, a cell surface protein, a receptor, a hapten, a post translational protein modification, a peptide, a permeabilized cell, and a virus.

The present disclosure provides, in various embodiments, “biomolecule-binding proteins.” By way of example, a biomolecule-binding protein can be, without limitation, a protein or protein domain, including an antibody, a nanobody, a T cell receptor, a B cell receptor, an antibody mimetic (e.g. DARPins, monobodies, affimers, alphabodies), MHC complex I and II, peptide binding domains (for instance SH3 domains), a B cell receptor, polypeptides, nucleic acid binding domains, lectins, pilins, cell receptor proteins (for instance toll like receptors or GPCRs), viral spike proteins, viral capsid proteins, including any fusion constructs, complexes, variants incorporating non-natural amino acids, or functional fragments of any of the aforementioned molecules, DNA, RNA and aptamers.

The disclosure also provides “binding domains” that are used in various aspects of the present disclosure. Binding domains, as used herein, refers to a molecule that can be encoded in RNA or DNA (e.g., to be part of an expression construct as described herein) that can spontaneously form a strong, non-covalent complex or a covalent complex (e.g., with a “binding partner.” The binding domains can be a protein, a polypeptide, a peptide, a non-natural amino acid, an aptamer, a nucleic acid sequence, a nucleoside analog, or a functional fragment thereof. Binding domains may also include, for example, covalent peptide tags, or pilin-derived proteins and peptides. The HUH-tag is an example of a protein that can form covalent bonds with a target DNA sequence (See, e.g., Lovendahl, K. N., et al., Journal of the American Chemical Society 139: 7030-7035 (2017)). FimGt/DsF is an example of an extremely stable non covalent pilin-derived protein/peptide tag that is contemplated (Giese, C.; et al., Angewandte Chemie International Edition 55: 9350-9355 (2016)). Additional embodiments and examples include, without limitation, Spycatcher protein and Spytag peptide (See, e.g., Zakeri, B., et al., Proceedings of the National Academy of Sciences 109: E690-E697 (2012), and Keeble, A. H.; et al., Proceedings of the National Academy of Sciences 116: 26523-26533 (2019)), streptavidin protein, neutravidin protein, DogTag and SnoopTag (See, e.g., Veggiani, G., et al., Proceedings of the National Academy of Sciences 113: 1202-1207 (2016)), IsopepTag (See, e.g., Zakeri, B. and Howarth, M., Journal of the American Chemical Society 132: 4526-4527 (2010)), SdyTag peptides and their cognate Catcher proteins (See, e.g., Tan, L. L., et al., PLOS ONE 11: e0165074 (2016)), split proteins such as split GFP, proteins derived from bacterial pilins, Halo-tags (See, e.g., Los, G. V., et al., ACS Chemical Biology 3: 373-382 (2008), and Koker, T., et al., Scientific Reports 8:5344 (2018)), Snap-tag (See, e.g., Juillerat, A., et al., Chemistry & Biology 10: 313-317 (2003)), Clip-tag (See, e.g., Gautier, A., et al., Chemistry & Biology 15: 128-136 (2008)), split inteins, and nucleic acids, or functional fragments of any of the above.

The terms “polynucleotide” and “nucleic acid” and “target nucleic acid” refer to a polymer composed of a multiplicity of nucleotide units (ribonucleotide or deoxyribonucleotide or related structural variants) linked via phosphodiester bonds. A polynucleotide or nucleic acid can be of substantially any length, typically from about six (6) nucleotides to about 10⁹ nucleotides or larger. Polynucleotides and nucleic acids include RNA, cDNA, genomic DNA. In particular, the polynucleotides and nucleic acids, is used herein to refer to a binding moiety used in the methods described herein and/or as a target of the methods described herein (e.g., a target whose location and sequence is determined by practicing the methods described herein). In some embodiments, the nucleic acid is rRNA, tRNA, mRNA, or mtRNA.

The term “oligonucleotide” refers to a polynucleotide of from about six (6) to about one hundred (100) nucleotides or more in length. Thus, oligonucleotides are a subset of polynucleotides. Oligonucleotides can be synthesized manually, or on an automated oligonucleotide synthesizer (for example, those manufactured by Applied BioSystems (Foster City, CA)) according to specifications provided by the manufacturer or they can be the result of restriction enzyme digestion and fractionation.

The term “protein” or “protein of interest” (e.g., as it relates to a (target) biomolecule or a “biomolecule-binding protein” or a “binding domain”) refers to a polymer of amino acid residues, wherein a protein may be a single molecule or may be a multi-molecular complex. The term, as used herein, can refer to a subunit in a multi-molecular complex, polypeptides, peptides, oligopeptides, of any size, structure, or function. It is generally understood that a peptide can be 2 to 100 amino acids in length, whereas a polypeptide can be more than 100 amino acids in length. A protein may also be a fragment of a naturally occurring protein or peptide. The term protein may also apply to amino acid polymers in which one or more amino acid residues is an artificial chemical analogue of a corresponding naturally occurring amino acid. A protein can be wild-type, recombinant, naturally occurring, or synthetic and may constitute all or part of a naturally-occurring, or non-naturally occurring polypeptide. The subunits and the protein of the protein complex can be the same or different. A protein can also be functional or non-functional.

An “expression construct,” as used herein, generally refers to a nucleic acid molecule comprising the sequences necessary to produce a transcription product (e.g., a mRNA or structural RNA) and, optionally, a translation product (e.g., a protein or polypeptide). The expression constructs provided herein may optionally include, without limitation, promoters, including inducible promoters or bidirectional promoters, origins of replication, selectable markers, ribosome binding sites, transcription initiation sites, translation initiation sites, and/or multiple cloning sites. Expression constructs can be expression vectors or plasmids. In some embodiments, the expression construct comprises a linker nucleic acid sequence between the nucleic acid sequence encoding the biomolecule-binding protein, and the nucleic acid sequence encoding a binding domain. Further, the nucleic acid sequence encoding the biomolecule-binding protein and the nucleic acid sequence encoding a binding domain can be combined with additional domains (e.g., at any position within their coding sequences) to provide additional functionalities including, for example, multimerization domains.

In other embodiments, an expression construct may comprise a solid substrate carrying nucleic acid fragments that encode a protein or RNA and the necessary regulatory elements to allow transcription and or translation of the encoded construct if in contact with an in vitro transcription and or translation reaction mixture. In one embodiment, the solid substrate carries multiple copies of the same nucleic acid fragment to enhance transcription and or translation. The solid substrate may contain additional nucleic acid sequences that modulate the expression reaction or modify the expression construct. In some embodiments, the solid substrate may be replaced by a droplet containing many copies of the same nucleic acid fragment.

In some embodiments, the expression constructs may comprise barcodes. The DNA barcode can be used as a molecular hash identifier of the biomolecule-binding protein sequence instead of its real sequence (or parts of it). The correlation of the barcode hash with the true coding sequence can be established through, for example, a sequencing step which links the barcode to the coding sequence. Subsequently, the binding domain can bind the short barcode instead of the coding sequence while encoding the same information.

In some embodiments, the “solid substrate” is a bead, a hydrogel bead, a microarray, a cell, a fixed cell, a cell fragment, a virus, a bacteriophage, a protein complex, a ribosome, a microparticle, a nanoparticle, a micelle, a liposome, a droplet, or a polymer.

Generally, other nomenclature used herein and many of the laboratory procedures in cell culture, molecular genetics and nucleic acid chemistry and hybridization, which are described below, are those well-known and commonly employed in the art. (See generally Ausubel et al. (1996) supra; Sambrook et al, Molecular Cloning: A Laboratory Manual, Second Edition, Cold Spring Harbor Laboratory Press, New York (1989), which are incorporated by reference herein). Standard techniques are used for recombinant nucleic acid methods, polynucleotide synthesis, preparation of biological samples, preparation of cDNA fragments, isolation of mRNA and the like. Generally enzymatic reactions and purification steps are performed according to the manufacturers' specifications.

“Detecting” or “determining” or “measuring” as used herein generally means identifying the presence of a target, such as a target nucleic acid or protein or biomolecule. In various embodiments, detection signals are produced by the methods described herein, and such detection signals may be optical signals which may include but are not limited to, colorimetric changes, fluorescence, turbidity, and luminescence. Detecting, in still other embodiments, also means quantifying a detection signal, and the quantifiable signal may include, but is not limited to, transcript number, amplicon number, protein number, and number of metabolic molecules. In this way, sequencing or bioanalyzers are employed in certain embodiments.

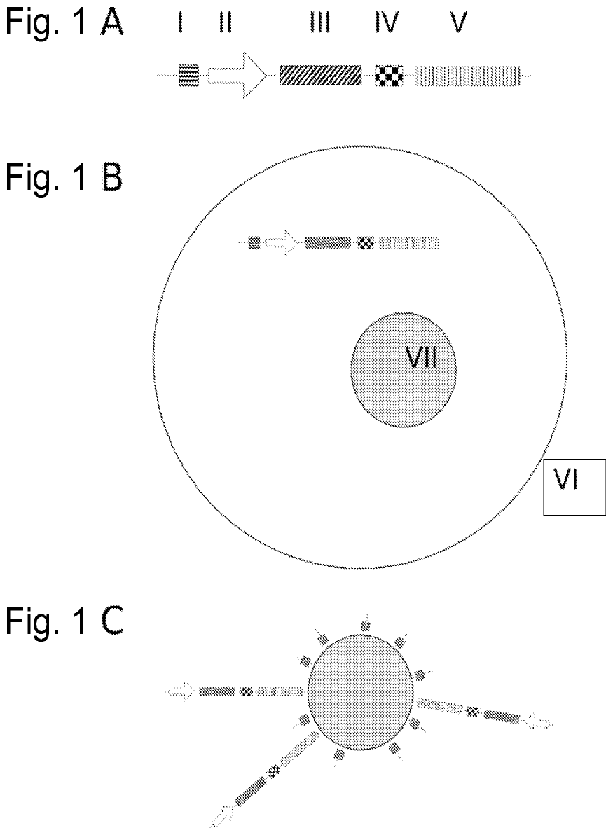

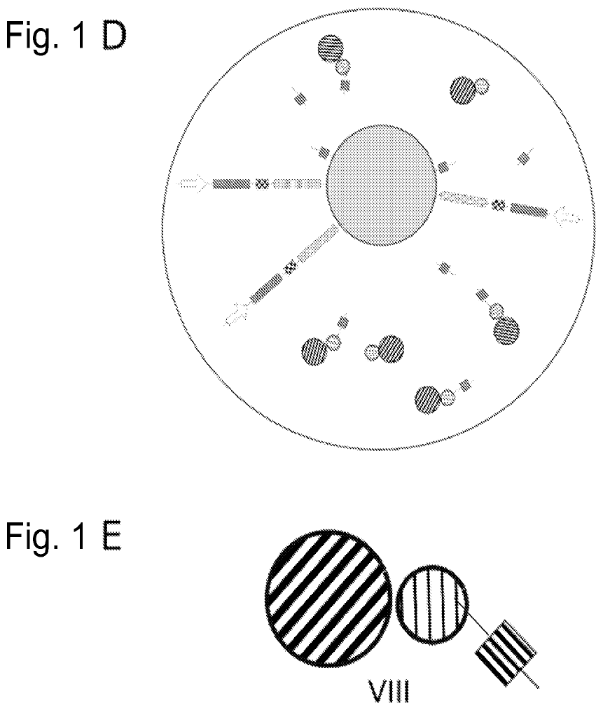

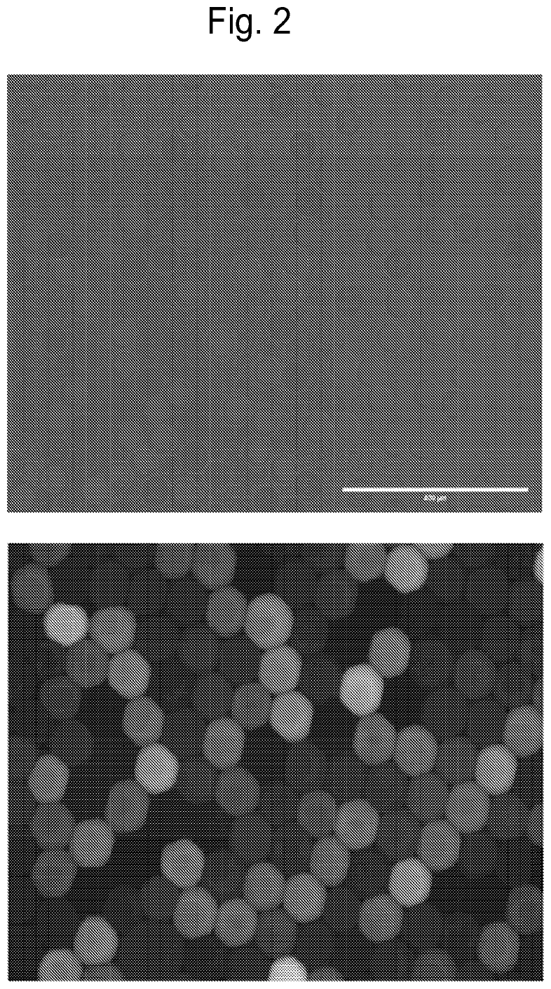

An exemplary workflow is as follows. An exemplary workflow is also shown in FIG. 1.

1. A diverse set of genes (or other nucleic acid fragments) that encode a diverse set of proteins or protein variants is obtained through any known method (e.g. full chemical synthesis, targeted or random mutagenesis of a backbone or obtained from nature). Genes encoding molecular binders, such as antibodies, nanobodies and other antibody mimetics or T-cell receptors, MHC complexes are contemplated in various embodiments of the present disclosure.

2. Genes or nucleic acid fragments thereof are placed in a suitable expression construct consisting of, for example, a promoter (e.g., T7) and a binding domain that is capable of forming covalent or strong non-covalent links with a partner molecule under suitable reaction conditions (e.g., streptavidin domain and biotin, Spycatcher protein and Spytag peptide, DNA binding domain and DNA tag). In some embodiments, the genes and the binding domain may optionally be connected through a linker which might provide additional functionality such as multimerization. Furthermore, the construct may carry a unique DNA barcode that allows identifying the gene library member placed in the construct.

3. These expression construct (or constructs) are next attached to a solid substrate such as a hydrogel bead in such a way that most beads carries a large number of identical copies from one to several gene constructs. Instead of a bead, in one embodiment the solid substrate is a support such as a spatial location on a microarray. This can be achieved, in one embodiment, by encapsulating beads with suitable primers and a single construct copy from the library, and performing a digital PCR that uses immobilized primers on the bead. The bead also carries a nucleic acid barcode sequence that can be used to infer the identity of the gene or gene fragments on the bead. This barcode sequence is modified with the corresponding binding partner molecule of the binding domain such that, if the construct is expressed as protein, it forms a strong interaction with the barcode molecules on the bead.

4. Modified beads are thus encapsulated in droplets (or otherwise segregated, e.g., a virtual confinement such as spots on a microarray that are not separated by a physical barrier but through a gap large enough to make molecule exchange unlikely) together with an in vitro transcription translation mix (or other cell free expression mixtures). In one embodiment, the mix transcribes a DNA construct into RNA and translates the RNA into protein. This causes the binding domain to fold and to bind to the binding partner and barcode structure. Because beads are separated physically (or virtually), the expressed constructs will thus label themselves predominantly with the barcode that corresponds to their bead of origin and hence can be linked to their genetic blue print. After the aforementioned expression, the barcodes can then be released from the solid support, thereby releasing the now barcoded protein library. This protein library is now functionally equivalent to the barcoded antibodies (for example as described in the DAb-seq method; Demaree, B., et al., Nature Communications. 2021. PMID: 33707421).

5. The expressed protein library can then be used to stain cells (e.g., analogous to DAb-seq). Since these libraries can have up to 10⁷-10⁸ members, this workflow provides a very detailed phenotypic spectrum of single cells which can reveal unexpected signatures such as, for example, tumor associated signatures. Additional applications of the workflow will also be appreciated by those of skill in the art, including, for example, cross-reacting the library of potential target proteins with a similarly prepared library of binders to identify cognate pairs and their binding affinities.

As will be appreciated by those of skill in the art, the methods provided herein contemplate segregation of the various constructs and complexes, for example in a microarray, rather than in, for example, a droplet.

In some embodiments of the present disclosure, a method is provided for preparing an expression construct library that uses an in vitro compartmentalization of an amplified DNA library such that each compartment receives many copies of the same DNA sequence. In this way, the present disclosure contemplates using a gene library with digital PCR in compartments with a solid substrate to create isogenicaly covered solid substrates. This provides a discrete unit (which can be manipulated easily) with high local DNA concentration, suitable for protein expression. The solid substrate also enables preparing the self-labeling reaction because excess reagents that would otherwise inhibit subsequent steps can be washed away (in the Example provided herein, excess spytag peptide would interact with the spycatcher protein and prevent barcoding if it couldn't be washed away without solid support).

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a conformation switching probe” includes a plurality of such conformation switching probes and reference to “the microfluidic device” includes reference to one or more microfluidic devices and equivalents thereof known to those skilled in the art, and so forth. It is further noted that the claims may be drafted to exclude any element, e.g., any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible. This is intended to provide support for all such combinations.

### EXAMPLES

### Example 1

### Construction of a Barcoded Nanobody Library

This Example-describes the construction of a library of DNA encoded nanobodies and uses thereof. FIG. 1 shows exemplary key steps in the workflow.

First, an expression construct is fabricated by creating polyacrylamide beads (6% w/v, 1:30 N, N′-Methylenebisacrylamide: acrylamide) with a diameter of 50 μm, as described previously (Delley, C. L. and Abate, A. R. (2021) Scientific Reports 11:10857). Two primers are copolymerized with the bead: primer A: Acrydite-CCUCCTACTCTGACGTCGNNNNNNGGTACCTTGTCCCCA (SEQ ID NO: 1) and primer B: Acrydite-ACAATAAGCTCTATCCACGATATAGTTCCTCCTTTCAGCAAAAAAC (SEQ ID NO: 2). Primer A harbors a uracil base, an unique molecular identifier (UMI) and is complementary to the constant region downstream of the CD3 loop. Primer B is complementary to the T7 terminator sequence. After preparation of the hydrogel beads, a microfluidic bead reinjector is used to encapsulated the beads in water-in-oil droplets together with PCR reagents (NEB Q5 ultra II), 0.4 μM of primer C: Azide-ACCGCGGTCTATTACTG (SEQ ID NO: 3) (complementary to the region upstream of CD3) and 0.4 μM primer D: GCGAAATTAATACGACTCACTATAGG (SEQ ID NO: 4) (T7 promoter sequence) and a DNA vector library encoding different nanobodies which are fused in frame to the spycatcher protein (Keeble, A. H., et al., Proceedings of the National Academy of Sciences 116: 26523-26533 (2019)) (FIG. 1A). Between 1000 to 10⁸ compartments can be formed this way. The DNA vector concentration is chosen such that on average 1 DNA molecule is present in each droplet (FIG. 1B). The number of different nanobodies present in all drops is hence Poisson distributed with a lambda of 1. The resultant emulsion is then thermocycled with the following program: 98° C. 45s 50 repeats of 98° C. 15s, 65° C. 1:15 min, then 65° C. 5 min, hold at 12° C. This emulsion PCR amplifies two constructs: the nanobody spycatcher gene together with the flanking T7 promoter elements (primer B and D) and the CD3 loop section of the nanobody (primer A and C). Because primer A and B are covalently linked to the hydrogel, the amplified DNA fragments remain also bound to the hydrogel bead (FIG. 1C). Because most droplets (one third) received exactly 1 plasmid copy, these beads are covered with isogenically with a T7 polymerase transcribable nanobody-spycatcher gene and the corresponding non transcribable short CD3 loop DNA sequence. The other two thirds of the beads either remain empty, or are covered with more than one distinct nanobody gene. due to the poisson distributed DNA loading into droplets. Because the CD3 loop contains very high sequence diversity in antibodies and nanobodies and is most important for target specificity, the corresponding short DNA sequence can be used as a barcode that uniquely identifies the nanobody gene. After the PCR the emulsion is broken, the beads harvested, and washed. Next the Azide moiety of primer C is functionalized with the spytag peptide: DBCO-CRGVPHIVMVDAYKRYK (SEQ ID NO: 5), through click chemistry using a Dibenzocyclooctyne-amine (DBCO) group and excess washed away. This step completes the construction of the expression construct.

To express the nanobody library, the prepared expression construct is reinjected in water-in-oil droplets using a microfluidic device together with an in vitro transcription translation (IVTT) mix (NEB PURExpress), a RNAse inhibitor (NEB Murine RNAse inhibitor) at the appropriate concentrations and incubated at 30° C. for 4 h (FIG. 1D). The reaction mix transcribes and translates the nanobody-spycatcher fusion construct from the DNA template on the beads. The expressed proteins fold which causes the spycatcher protein to bind to the spytag peptide and form an isopeptide bond, thereby immobilizing the expressed nanobodies on the beads through the CD3 DNA fragment which serves as identifying barcode. Next, the emulsion is again broken, the beads collected and the reaction mixture washed away. Finally, the expressed and barcoded nanobody library is released from the bead by cleavage of the primer A with the USER enzyme mix (NEB) and the beads removed by a strainer. This results in a barcoded nanobody library (FIG. 1E), which can have up to 10⁸ different sequences (as many as compartments) and each sequence present at about 10⁹ copies.

The barcoded nanobody library can then be used to stain cells analogously to antibodies used in for instance a DAb-seq experiment (Demaree, B., et al., Nature Communications 12:1583 (2021)). In contrast to a regular DAb-seq experiment that uses a selected panel of antibodies, the here described nanobody library can stain any known and unknown epitope on the target cell by virtue of its large sequence diversity.

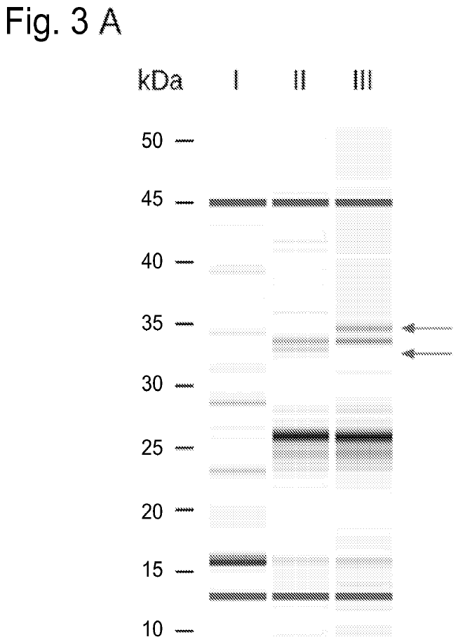

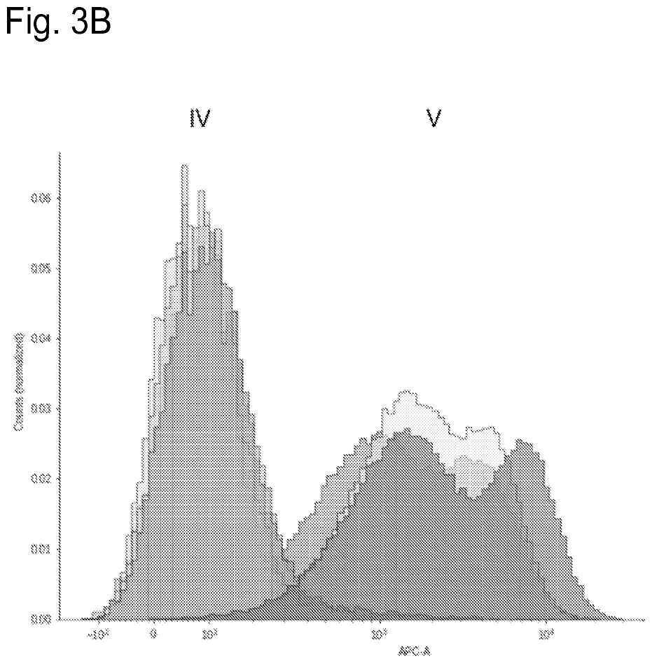

As shown in FIG. 2, an expression construct prepared as described herein yields correctly-folded GFP. As shown in FIG. 3, self-labeling of molecular binders with DNA barcodes as described herein occurs in droplets and is capable of labeling cells.

The various embodiments described above can be combined to provide further embodiments. All U.S. patents, U.S. patent application publications, U.S. patent application, foreign patents, foreign patent application and non-patent publications referred to in this specification and/or listed in the Application Data Sheet are incorporated herein by reference, in their entirety. Aspects of the embodiments can be modified if necessary to employ concepts of the various patents, applications, and publications to provide yet further embodiments.

These and other changes can be made to the embodiments in light of the above-detailed description. In general, in the following claims, the terms used should not be construed to limit the claims to the specific embodiments disclosed in the specification and the claims but should be construed to include all possible embodiments along with the full scope of equivalents to which such claims are entitled. Accordingly, the claims are not limited by the disclosure.

1. A method of identifying a biomolecule from a single cell and identifying a binding partner of said biomolecule, said method comprising:
(a) preparing an expression construct comprising (i) a nucleic acid sequence encoding a biomolecule-binding protein, and (ii) a nucleic acid sequence encoding a binding domain capable of binding to a binding partner;
(b) attaching the expression construct of (a) to a solid substrate, thereby forming an expression construct-substrate complex, wherein said solid substrate comprises a nucleic acid barcode and the binding partner, thereby forming an expression construct-substrate complex;
(c) isolating the expression construct-substrate complex of (b);
(d) incubating the expression construct-substrate complex under conditions that allow (i) transcription and translation of the biomolecule-binding protein and the binding domain, and (ii) binding of the binding domain to the binding partner on the solid substrate, thereby labelling the biomolecule-binding protein with the nucleic acid barcode;
(e) releasing the biomolecule-binding protein of (d) from the solid substrate and contacting the biomolecule-binding protein with a sample comprising a plurality of cells under conditions that allow binding of the biomolecule-binding protein to a biomolecule on a single cell;
(f) encapsulating a single cell bound by the biomolecule-binding protein of (e) in a droplet; and
(g) sequencing the nucleic acid barcode of the biomolecule-binding protein;
thereby identifying a biomolecule from a single cell and a binding partner of said biomolecule.

2. The method of claim 1 wherein step (c) comprises encapsulating in a droplet.

3. The method of claim 1, wherein (i) the nucleic acid sequence encoding the biomolecule-binding protein and/or (ii) the nucleic acid sequence encoding the binding domain is a DNA sequence, and wherein (i) and (ii) are operably linked to a promoter.

4. The method of claim 1, wherein the nucleic acid sequence encoding the biomolecule-binding protein and/or the nucleic acid sequence encoding the binding domain is an RNA sequence.

5. The method of claim 1, wherein the biomolecule-binding protein is an antibody, a nanobody, a T cell receptor, a B cell receptor, or an antibody mimetic.

6. The method of claim 1, wherein the binding domain is a protein, a polypeptide, a peptide, a non-natural amino acid, an aptamer, a nucleic acid sequence, a nucleoside analog, or a functional fragment thereof.

7. The method of claim 6, wherein the binding domain is a peptide.

8. The method of claim 7, wherein the binding domain is a Spycatcher peptide and the binding partner is a Spytag peptide.

9. The method of claim 7, wherein the binding domain is streptavidin or neutravidin or fragment thereof and the binding partner is biotin, a biotin analog or peptide with affinity for streptavidin and neutravidin.

10. The method of claim 1, wherein the expression construct further comprises a unique DNA barcode.

11. The method of claim 1, wherein the expression construct further comprises a linker nucleic acid sequence between the nucleic acid sequence encoding the biomolecule-binding protein, and the nucleic acid sequence encoding a binding domain.

12. The method of claim 1, wherein the expression construct is a plasmid, a DNA fragment, an RNA or an mRNA.

13. The method of claim 1, wherein the solid substrate is a bead, a hydrogel bead, a microarray, a cell, a fixed cell, a cell fragment, a virus, a protein complex, a ribosome, a microparticle, a nanoparticle, a micelle, a liposome, a droplet, or a polymer.

14. The method of claim 1, wherein the sample comprising the plurality of cells is a tumor sample, a tissue sample, a blood sample, an environmental sample, a microbial sample, a bacterial sample, a viral sample, or mixtures thereof.

15. The method of claim 1, wherein the biomolecule is an antigen, a tumor antigen, a cell surface protein, a receptor, a hapten, a post translational protein modification, a peptide, a permeabilized cell, or a virus.

16. A method of determining a phenotype of a single cell comprising:
(a) preparing an expression construct comprising (i) a nucleic acid sequence encoding a biomolecule-binding protein, and (ii) a nucleic acid sequence encoding a binding domain capable of binding to a binding partner;
(b) attaching the expression construct of (a) to a solid substrate, thereby forming an expression construct-substrate complex, wherein said solid substrate comprises a nucleic acid barcode and the binding partner, thereby forming an expression construct-substrate complex;
(c) isolating the expression construct-substrate complex of (b);
(d) incubating the expression construct-substrate complex under conditions that allow (i) transcription and translation of the biomolecule-binding protein and the binding domain, and (ii) binding of the binding domain to the binding partner on the solid substrate, thereby labelling the biomolecule-binding protein with the nucleic acid barcode;
(e) releasing the biomolecule-binding protein of (d) from the solid substrate and contacting the biomolecule-binding protein with a sample comprising a plurality of cells under conditions that allow binding of the biomolecule-binding protein to a biomolecule on a single cell;
(f) encapsulating a single cell bound by the biomolecule-binding protein of
(e) in a droplet; and
(g) sequencing the nucleic acid barcode of the labeled biomolecule-binding protein;
thereby determining a phenotype of a single cell.

17. An expression construct-substrate complex comprising:
(a) at least one expression construct comprising (i) a nucleic acid sequence encoding a biomolecule-binding protein, and (ii) a nucleic acid sequence encoding a binding domain capable of binding to a binding partner; and
(b) a solid substrate comprising a nucleic acid barcode and the binding partner.
